(19) United States
(12) Patent Application Publication
(10) Pub. No.: US 2009/0192538 A1
(43) Pub. Date: Jul. 30, 2009

(54) SAFETY SCALPEL HANDLE

(76) Inventors: Dan Sandel, Chatsworth, CA (US); Ruane Jeter, Chatsworth, CA (US)

Correspondence Address: PERKINS COIE LLP, POST OFFICE BOX 1208, SEATTLE, WA, 98111-1208, US

(21) Appl. No.: 12/019,494

(22) Filed: Jan. 24, 2008

(51) Int. Cl. A61B 17/32; A61B 17/14

(52) U.S. Cl. 606/167; 606/182

(57) ABSTRACT

A scalpel handle includes a sliding shield to block the blade tip or surround the blade when the scalpel is not in use. The shield is advantageously slidable from an extended position to a withdrawn position, wherein the shield is substantially within the handle. Consequently, the user's hand grasps only onto the handle, and not the shield. This helps to provide a more solid feel, similar to a conventional scalpel handle. When in the withdrawn position, the shield avoids interfering with cutting or puncturing blade movements.

### BACKGROUND OF THE INVENTION

Conventional scalpels used in surgical procedures typically have a cutting blade attached to a metal handle. The blade is attached to the handle via a slot formed in the blade and a corresponding track or lug protruding from the handle. Especially during surgery, the sharp edge of the blade may be a hazard to nurses and surgeons. Accidental cutting or puncturing (or so-called “sharps accidents”) are a serious risk in an operating room. If the surgical glove and skin of the surgeon or nurse is accidentally cut via a scalpel blade, there is a risk of transmission of blood borne infectious diseases, and loss of sterility.

To better avoid sharps accidents, various safety scalpels have been proposed. Safety scalpels typically have a blade that slides in and out of a handle, a shield that slides over the blade, or a blade within a cartridge. With proper use of these types of safety scalpels, the sharp edge of the blade is not exposed, except during actual use of the scalpel. However, many surgeons object to these types of scalpels for various ergonomic or performance reasons.

Many surgeons who have been trained with conventional scalpels, object to using safety scalpels because the grip, balance, weight, or overall feel of the safety scalpel handle is different from a conventional scalpel handle. In general, the surgeon's visual sight lines to the blade of a safety scalpel are also not as good as with a conventional scalpel, because the front end of the safety scalpel handle is wider, and often also taller, than the front end of a conventional scalpel handle. This difference can also limit the surgeon's ability to make plunging or puncturing incisions using a safety scalpel.

With many safety scalpels, the blade cannot be changed. Consequently, the hospital must buy and stock multiple safety scalpels having blades of different sizes and shapes, as may be preferred by the surgeon for a specific operation. This increases cost and complexity. Scalpel blades are also typically either carbon steel or stainless steel. Carbon steel blades tend to have a sharper edge, but are prone to rusting. Due to limitations in packaging, safety scalpels tend to be provided only with stainless steel blades, which do not readily rust. Some surgeons consequently will not use safety scalpels because they prefer the sharper edge of a carbon steel blade.

Even with safety scalpels that allow the blade to be changed, disadvantages remain. Specifically, since the size and shape of the front end of the safety scalpel handle is different from a conventional scalpel handle, conventional blade changing techniques and blade removing devices may not work with safety scalpels. As a result, surgeons, and especially nurses (who more often handle changing the blade) may be resistant to using safety scalpels. Accordingly, engineering challenges remain in providing an improved safety scalpel handle.

### SUMMARY OF THE INVENTION

A safety scalpel handle may be provided with an interior metal element or insert having front and back sections. A handle body may be provided around the insert. The front end of the insert extends out from the front of the handle body. The front end of the insert may be virtually identical in size and shape to a conventional metal scalpel handle. A shield is advantageously slidable within the handle, between an extended position, where the shield surrounds or covers the point and edge of a blade on the front end of the insert, to a retracted position, where the blade and front end of the insert are exposed. With the shield in the retracted position, the front of the safety scalpel generally has the same size, shape, visual sight lines, and performance characteristics of a conventional scalpel, while also providing the blade shielding features of a safety scalpel. Other and further objects and advantages will appear hereinafter. The invention resides as well in subcombinations of the features described.

### BRIEF DESCRIPTION OF THE DRAWINGS

In the drawings, the same element number indicates the same element in each of the views:

FIG. 1 is front, top and left side perspective view of the present scalpel handle, with a blade installed and the blade shield shown in the withdrawn position.

FIG. 2 is front, top and right side perspective view of the present scalpel handle, with a blade installed and the blade shield shown in the withdrawn position.

FIG. 3 is front, top and left side perspective view of the present scalpel handle, with a blade installed and the blade shield shown in the extended position.

FIG. 4 is front, top and right side perspective view of the present scalpel handle, with a blade installed and the blade shield shown in the extended position.

FIG. 5 is front, top and left side perspective view of the present scalpel handle, without a blade and the blade shield shown in the withdrawn position.

FIG. 6 is front, top and right side perspective view of the present scalpel handle, without a blade and the blade shield shown in the withdrawn position.

FIGS. 7A and 7B are exploded front, top and side perspective views of the scalpel handle shown in FIGS. 1-6.

FIG. 8 is front, top and right side perspective view of the insert shown in FIG. 7.

FIG. 9 is front, top and right side perspective view of an alternative insert.

FIG. 10 is front view of the scalpel handle shown in FIGS. 1-4.

FIG. 11 is a top view of the scalpel handle shown in FIG. 6.

### DETAILED OF DESCRIPTION OF THE DRAWINGS

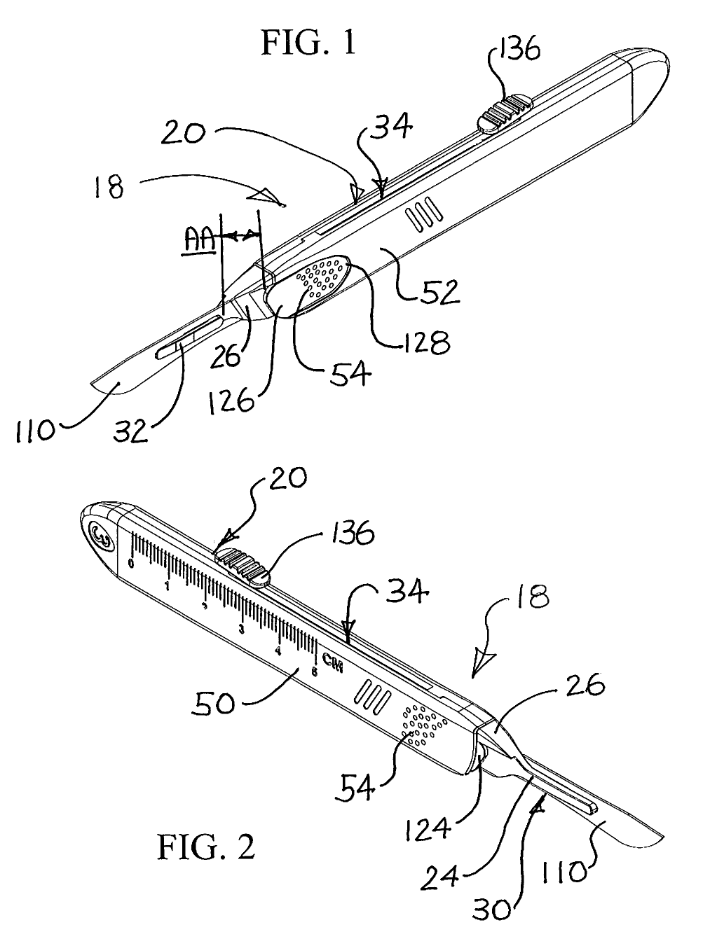

As shown in FIGS. 1 and 2, a scalpel handle 20 has a handle or grip portion formed by a first or right side section 50 joined to a second or left side section 52. The side sections 50 and 52 may be made of a plastic material. A measuring scale, such as an inches or millimeter scale, may be molded into or printed on the outside surface of either section 50 or 52. Generally, a friction surface 54 is provided near the front end of both sections 50 and 52. The friction surface 54 may be formed by a pattern of dots, dimples, ridges, recesses, or knurled areas. The friction surface 54 provides a slip resistance gripping surface for the user near the front of the scalpel handle 20. The handle 20 together with a blade 110 form a scalpel 18.

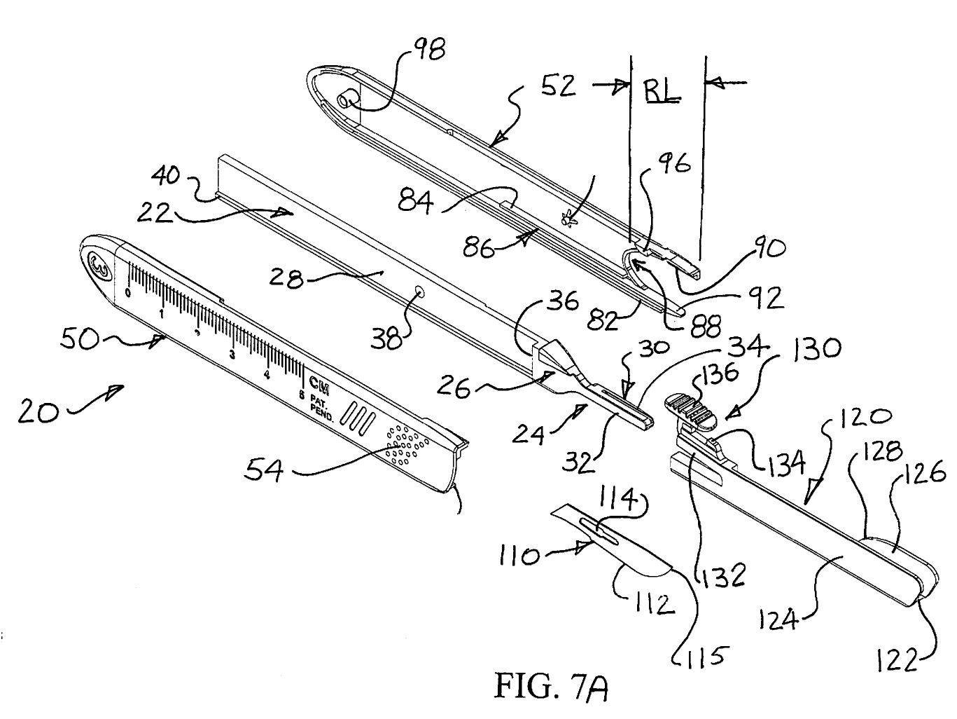

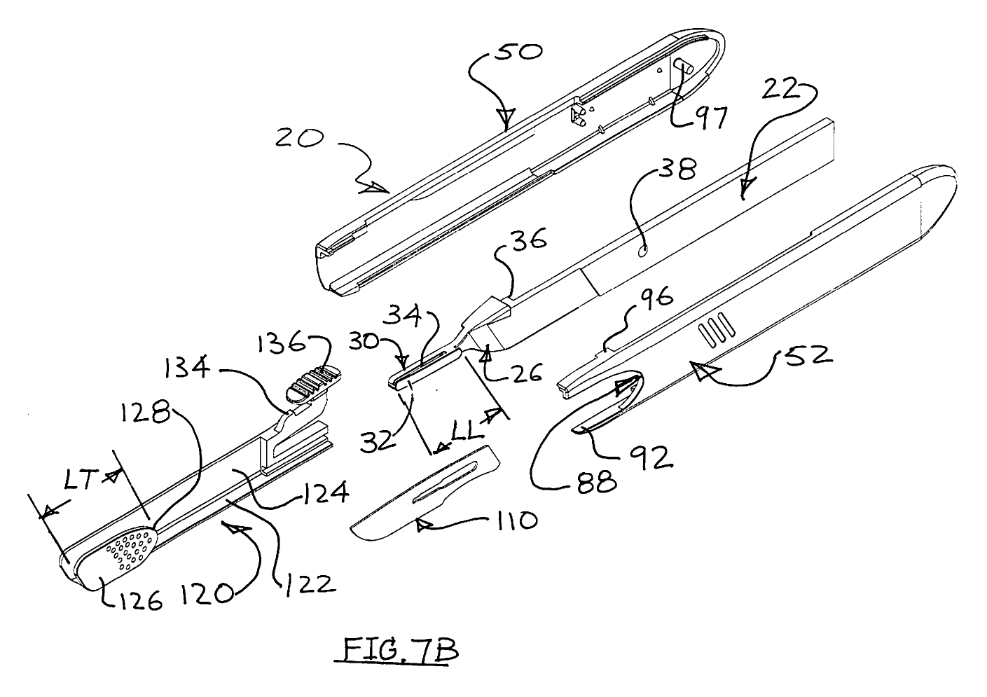

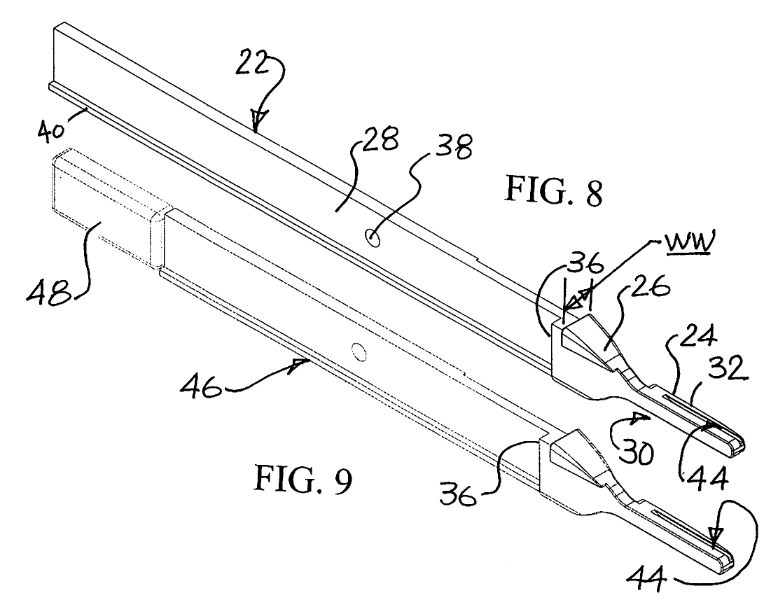

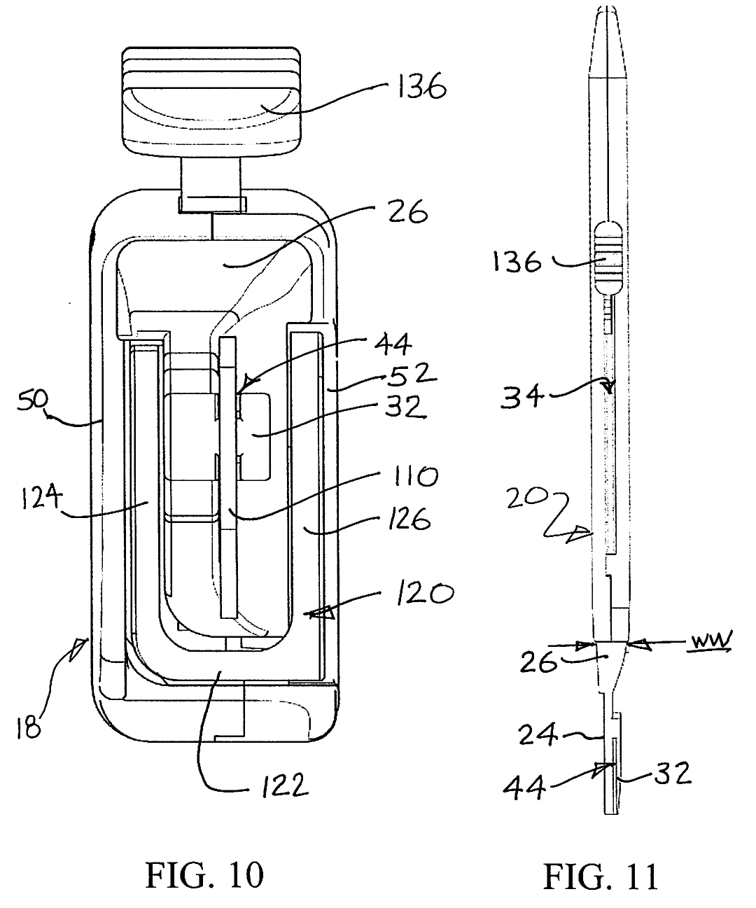

Turning to FIG. 7B, an insert 22 in substantially enclosed between the side sections 50 and 52. The insert 22, which may be made of a metal, such as steel or aluminum. The insert 22 may be formed as a single solid piece, as shown in FIG. 8. Alternatively, an insert 46 having a separate back end 48, or other additional separate components, may be used. The insert 22 has a front section 24, a transition section 26 and a back section 28. The transition section 26 reduces down from the back section 28 which is generally in the form of a rectangular prism, to the front section, which has a relatively elongated and narrow blade holder 30. The transition section generally extends forward from a front wall 36 to the blade holder 30.

Referring still to FIGS. 7A and 7B, the blade holder 30 at the front end of the insert 22 has a blade slot 44 in a lug 32 adapted to hold a scalpel blade 110. A lip 40 extends generally perpendicularly outwardly at the bottom of the back section 28. The left side section 52 can include a rib 84 extending parallel to a bottom wall 82, with a slot 86 formed between them. The right side section 50 may have a similar design. The left and right sections also have top, bottom, and back surfaces, so that when assembled together, they substantially enclose the entire back section 28 of the insert 22, as shown in FIGS. 1 and 2.

The left side 52 may include posts 98 and 100, or other fasteners or attachment devices, to attaché the left side 52 to the right side 50. In the specific design shown, the front post 100 passes through a clearance hole 38 in the back section 28 of the insert 22 and engages the right side 50. A rear peg 97 on the right side section 50 may be located behind the back end of the insert 22 and engage into a post 98 on the left side section 52. The lip 40 on one or both sides of the insert 22 may extend into the slot 86 formed on the inner surface of the left side section 50 and/or the right side section 50, to secure the insert in place, and to make the handle 20 of the scalpel 18 more rigid. The front end of the right side section 50 may seat against the front wall 36 to further prevent movement between the insert 22 and the left side section 50. A similar feature may be provided for the left side section 52. A contoured or curved recess 88 can be formed between a top prong 90 and a bottom prong 92 extending forward from the front end of the left side section 52.

Referring still to FIGS. 7A and 7B, a sliding shield 120 includes a side plate 124 and a tab plate 126 attached on opposites sides of a bottom plate 122. The side plate 124 is spaced apart from and generally parallel to the tab plate. The side plate 124 and the tab plate 126 are generally both perpendicular to the bottom plate 122. The tab plate 126 may have a rear edge 128 that is shaped to fit into and match with the recess 88. The tab plate 126 has a length LT preferably equal to, or greater than, the length LL of the lug 32, as indicated in FIG. 7B. The recess 88 has a length or depth RL, shown in FIG. 7A, typically greater than the length LL of the lug 32 shown in FIG. 7B.

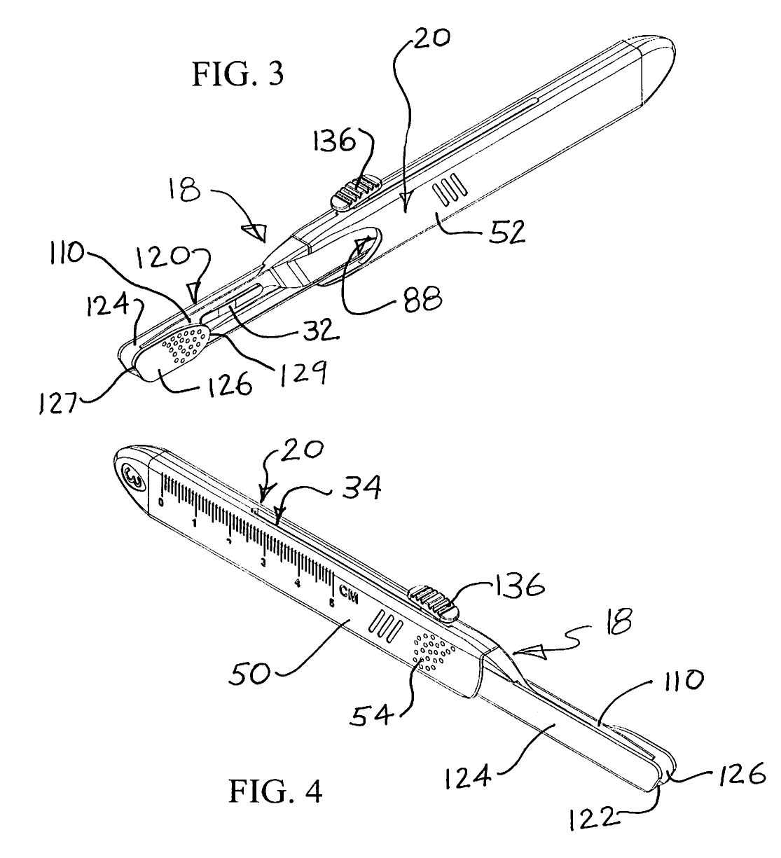

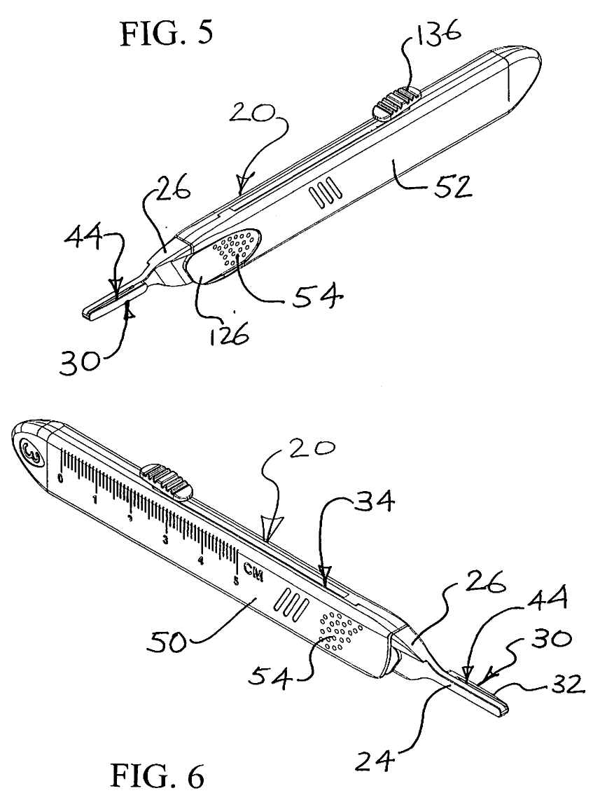

A latch may optionally be provided at the back end of the shield 120. The latch may include a button 136 and a cam 134 on a lever 132. The shield 120 may be molded as a single plastic piece. The shield may be made with a color, such as yellow or orange, that contrasts with the color of the handle, and with most commonly used surgical drapes and gowns, which are typically blue or green. As also shown in FIG. 7, a scalpel blade 110 having a cutting edge 112 and a blade slot 114, is used with the scalpel handle 20. FIGS. 1-4 show the scalpel handle 20 with a blade 110 installed on the blade holder 30. FIGS. 5 and 6 shown the scalpel handle 20 without any blade. In use, the scalpel handle 20 may be provided in a sterile condition within a package or container, alone or a component of a kit. Generally, the blade 110 may be provided separately and is installed just before use. However, for some applications, the scalpel handle 20 may be packaged with a blade already installed. The shield 120 will ordinarily be in the extended position, as shown in FIGS. 3 and 4. In this position, the edge 112 of the blade 110 is covered or blocked by the bottom plate 62 of the shield, as shown in FIGS. 3 and 4. The tip or point of the blade is also shielded between the side plate 124 and the tab plate 126. With the shield 120 in the extended position, the button 136 is positioned at the front end of a latch slot 34 formed in the top of the handle. The latch slot 34 may be formed by providing a half-slot recess in each of the sides 50 and 52, or by providing a slot opening in either of the sides 50 and 52.

Referring to FIG. 7A, with the shield fully extended, the cam 134 is in front of the forward latching block 96. This prevents the shield 120 from moving rearwardly, unless the button 136 is pressed down, to release the latch 130. The shield is generally left in the extended position, until the scalpel is in the surgeon's hand and ready for use in surgery. With the shield 120 extended, the sharp surfaces of the blade surrounded by the shield, to prevent accidental cutting or puncture. If the shield 120 is made of contrasting color, the users are provided with a strong visible cue that the shield is extended and the blade is covered.

As shown in FIG. 3, with the shield extended, the front edge 127 of the tab plate 126 is a few millimeters (e.g., 2-6 mm) in front of the tip 115 of the blade 110. The tab plate 126 is shorter than the side plate 124, so that the back edge 129 of the tab plate 126 is in front of, or generally aligned with the front of the lug 32. As a result, most of the left side surface of the blade is visible, even with the shield extended. This allows the user to determine the size, shape, style and condition of the blade, without the need to retract the shield.

When the scalpel 18 is ready for use, the button 136 is pressed down with the thumb. This moves the cam 134 down from front latching block 96. The user then draws the button 136 back, causing the shield 120 to move back from the position shown in FIGS. 3 and 4, to the position shown in FIGS. 1 and 2. This movement may also be performed with the thumb. As result, the entire release and movement of the shield may be performed with one hand.

As the shield 120 is moved back, it slides into the handle, between the side sections 50 and 52. The bottom plate 122 of the shield 120 may be positioned to slide within a plate slot formed on the inside front surface of the left side section 52. This can help to hold the shield into position and alignment, as the shield is moved rearwardly. In addition, is the back edge of the tab plate 126 is shaped to fit into the recess 88, the shield may be further supported when retracted into the handle. The length LT of the tab plate 126 generally may be about equal to, or greater than, the length of the lug 32.

With the shield is the withdrawn position, the blade 110 is fully exposed for surgery, as shown in FIGS. 1 and 2. The handle, as formed by the side sections 50 and 52, only covers the back section of the insert 22 (on which the blade is mounted). The transition section 26 and front section 24 of the insert are not affected by the handle. Accordingly, with the transition section 26 and the front section 24 are formed in the same size and shape as with a conventional metal scalpel handle, then the present scalpel handle 20 is able to substantially match the performance of a conventional metal scalpel, for example in terms of visibility of the blade, cutting angles, cutting depth, and clearance around the blade.

For example, the scalpel 18 can be used to make a deep puncture, in the same way as a conventional metal scalpel, because with the shield withdrawn, no part of the shield or handle interferes with use of the blade. The transition section 26 and the front section 24 may have the same size and shape as conventional scalpel handles, such as BD Bard-Parker handles, available from BD Medical, Franklin Lakes, N.J., or the Feather stainless steel scalpel handle available from Feather Safety Razor Co., Osaka, Japan. The specific design shown in the drawings has a front section and a transition section similar to a Feather No. 3 stainless steel handle.

Referring to FIG. 8, the transition section 26 typically has a maximum width WW (here across the top) of about 5, 6, 7 or 8 mm, with a nominal width of about 3, 4 or 5 mm. The transition section also has an un-occluded length AA, shown in FIG. 1, of at least about 5, 6, 7, 8, 9 or 10 mm. This un-occluded length is the dimension from the back end of the lug 32 to the front edge of the shield 120 or either side section 50 or 52 (with the shield fully retracted). In the design shown in the drawings, AA extends from the back end of the lug 32 to the front edge of the side plate 124 and the front edge of the tab plate 126, since the plates 124 and 126 extend slightly forward of the side sections 50 and 52, as shown in FIGS. 1 and 2. The un-occluded length is achieved by using an insert having a front section 24 and a transition section 26 similar to, or the same as, a conventional scalpel handle, and with the additional components (here the side sections 50 and 52 and the shield 120) located substantially or entirely behind the transition section 26. As an example, the Feather No. 3 handle has a maximum width WW of about 3 or 4 mm, and a dimension AA of about 5-6 mm. The transition section 26 may vary with different handle designs. In one instance, it may be measured as the section of the handle having a forward starting point where the width and/or height of the blade holder 30 is increasing, and a rearward ending point where the width and/or height of the handle reaches a maximum.

When the surgeon has completed use of the scalpel 18, or to temporarily shield the blade 110, the button 136 is pushed forward, to move the shield back to the extended position shown in FIGS. 3 and 4. The cam 134 is resiliently depressed, slides under the latching block 96 and then flexes back up. This locks the shield 120 again into the extended position. The only moving component on the scalpel handle is the shield 120, which includes the button 136. The blade 110 is directly attached to the insert 22, which is secured within the handle, between the side sections 50 and 52. As a result, the scalpel handle 20 provides a solid feel in the surgeon's hand, similar to a conventional metal scalpel handle.

The blade holder 30 advantageously matches conventional scalpel blade holders. Accordingly, as the scalpel handle is generally provided without a blade, as shown in FIGS. 5 and 6, the user's preferred blade may be installed using conventional techniques. Similarly, a blade on the scalpel 18 may be removed and replaced using conventional blade changing techniques. For example, since the front and transition sections 24 and 26 may have essentially the same dimensions as a conventional scalpel handle, blade removers such as described in Sandel, U.S. Pat. No. 4,318,473, incorporated herein by reference, may be used to remove the blade (as the shield and side sections do not interfere with such use). As a result, the present scalpel handle 20 provides safety features, but allows for use of conventional scalpel handling and blade changing methods. This provides a more acceptable alternative to many surgeons and nurses, in comparison to many existing safety scalpels which require alternative handling.

Thus, a novel scalpel handle has been shown and described. Various changes and substitutions may of course be made within the spirit and scope of the invention. The invention, therefore, should not be limited, except to the following claims and their equivalents.

1. A scalpel handle, comprising:
an elongated metal insert having a front section, a transition section and a back section;
a blade holder on the front section of the metal insert;
a blade attached to the blade holder, with the blade having a cutting edge;
a plastic body substantially enclosing only the back section of the metal insert, with the plastic body including a first elongated side piece and a second elongated side piece attached to the first side piece, with the metal insert between them, and one or more friction surfaces on the plastic body adjacent to the transition section of the metal insert;
a recess in the first side piece;
a channel formed on an inside surface of the first side piece of the plastic body, with the slot facing the inside surface of the second side piece;
a slot in the top of the plastic body extending substantially from the recess to an intermediate position;
a plastic shield comprising:
a bottom plate joined to a side plate, and with the bottom plate substantially perpendicular to the cutting edge of the blade, and
with the bottom plate at least partially within the channel;
a tab plate attached to the bottom plate at a front end of the shield,
with the tab plated substantially parallel to the side plate, and with the tab plate shorter than the side plate;
an actuator lever adjacent the back end of the shield, with part of the actuator lever extending up through the slot in the top of the plastic body;
the shield slidable into and out of the plastic body, from a first position where the shield is extended out of the plastic body and the side and bottom plates of the shield entirely overly the cutting edge of the blade, with the bottom plate facing the blade and substantially perpendicular to the blade, and the tab plate overlies only a tip section of the blade, leaving the rest of the blade visible, to a second position, where the shield is withdrawn at least partially into the handle, to expose the cutting edge of the blade, and the with tab plate engaged into the recess in the first side piece of the plastic body when the shield is in the second position.

2. The scalpel handle of claim 1 with the tab plate having a contoured back surface substantially matching the shape of the recess in the first side piece.

3. The scalpel handle of claim 1 with the shield and the plastic body having contrasting colors.

4. The scalpel handle of claim 1 further comprising a releasable locking device associated with the shield for locking the shield into the first position or in the second position.

5. The scalpel handle of claim 1 wherein the shield comprises a finger, an actuator button on the finger having at least one hook, and with the actuator button resiliently displaceable to engage or release the hook from a latch on the plastic cover.

6. The scalpel handle of claim 1 with the blade located substantially on a centerline of the plastic cover.

7. The scalpel handle of claim 1 wherein the tab plate is positioned in front of the blade holder when the shield is in the first position.

8. A scalpel handle, comprising:
an elongated metal insert having a front section, a transition section and a back section, with the transition section having a maximum width of about 5 mm;
a blade holder on the front section of the metal insert, including a lug;
a plastic body substantially enclosing only the back section of the metal insert, with the plastic body including a first elongated side piece and a second elongated side piece attached to the first side piece, with the metal insert between them;
a recess in the first side piece;
a plastic shield comprising:
a bottom plate joined to a side plate;
a tab plate attached to the bottom plate at a front end of the shield,
with the tab plated substantially parallel to the side plate, and with the tab plate shorter than the side plate;
an actuator adjacent the back end of the shield, with part of the actuator extending up through a slot in the top of the plastic body;
the shield slidable into and out of the plastic body, from a first position where the shield is extended out of the plastic body and at least portions of the side and bottom plates extend forward of the lug, to a second position, where the shield is withdrawn at least partially into the handle, and the tab plate engaged into the recess in the first side piece of the plastic body.

9. The scalpel handle of claim 8 wherein the shield is positioned substantially entirely behind the transition section, when in the second position.

10. The scalpel handle of claim 8 wherein a back end of the tab plate is located behind a front end of the blade holder, when the shield is in the first position.

11. The scalpel handle of claim 8 wherein the recess in the side piece has a length greater than the length of the lug.
